(19) United States
(12) Patent Application Publication
(10) Pub. No.: US 2007/0093461 A1
(43) Pub. Date: Apr. 26, 2007

(54) Effect of Loteprednol etabonate on vascular dysfunction

(75) Inventors: Afshin Shafiee, Rochester, NY (US); Dharmendra Jani, Fairport, NY (US); Stephen P. Bartels, Pittsford, NY (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/519,414

(22) Filed: Sep. 12, 2006

Related U.S. Application Data
(60) Provisional application No. 60/730,277, filed on Oct. 26, 2005.

(51) Int. Cl. A61K 31/573

(52) U.S. Cl. 514/179

(57) ABSTRACT

This invention relates to the effect of Loteprednol etabonate on vascular dysfunction in the back of the eye. More specifically, this invention relates to methods of modifying a pathogenic angiogenesis in the back of an eye of a patient, the method comprising administering to a patient in need thereof a pathogenic angiogenesis modifying amount of Loteprednol etabonate. Moreover, this invention relates to methods of modifying pathologic vascular permeability manifested as retinal edema. The method compromises administering to a patient an amount of LE sufficient to reduce retinal edema.

### PRIORITY CLAIMS TO PRIOR APPLICATIONS

This application claims priority to U.S. Provisional Application 60/730,277 filed Oct. 26, 2005 the contents of which are incorporated by reference herein.

### FIELD OF THE INVENTION

This invention relates to the effect of Loteprednol etabonate on vascular dysfunction in the back of the eye. More specifically, this invention relates to methods of modifying a pathogenic angiogenesis in the back of an eye of a patient, the method comprising administering to a patient in need thereof a pathogenic angiogenesis modifying amount of Loteprednol etabonate. Moreover, this invention relates to methods of modifying pathologic vascular permeability manifested as retinal edema. The method compromises administering to a patient an amount of LE sufficient to reduce retinal edema.

### BACKGROUND AND SUMMARY

Compounds classified as corticosteroids, such as triamcinolone, can effectively treat some forms of neovascularization such as corneal neovascularization. In general, corticosteroids have been unsuccessful in treating neovascularization of the posterior segment. In many patients, these compounds cause undesirable side effects. These adverse affects include elevations in intraocular pressure and the formation of, or acceleration of the development of, cataracts. Elevations in intraocular pressure are of particular concern in patients who are already suffering from elevated intraocular pressure, such as glaucoma patients. Moreover, a risk exists that the use of corticosteroids in patients with normal intraocular pressure will cause elevations in pressure that result in damage to ocular tissue. Since therapy with corticosteroids is frequently long term, i.e., several days or more, a potential exists for significant damage to ocular tissue as a result of prolonged elevations in intraocular pressure attributable to that therapy.

One approach to solving the foregoing problems has been to search for specific compounds which are effective in treating neovascularization without elevating intraocular pressure. Another approach has been to administer corticosteroids in conjunction with another drug to “block” or reduce the IOP elevating effects of the corticosteroids or to treat IOP elevation separately with another drug. A further approach has been to intravitreally inject corticosteroids to treat ocular neovascularization or retinal edema.

U.S. Pat. No. 5,646,136 discloses methods for treating angiogenesis, tumors, and ocular hypertension with steroids including cortienic acid.

There still exists a need for an improved method for treating and/or preventing retinal diseases with corticosteroids.

Loteprednol etabonate (LE) is a predictably metabolized steroid that is being used as a topical anti-inflammatory agent. We have discovered that LE also has an anti-angiogenic effect in the eye by inhibiting the formation of formation of VEGF (Vascular endothelial growth factor), a growth factor that stimulates new blood vessel growth and down regulating VEGF a potent endothelial cell specific mitogen and ICAM-1 (intracellular adhesion molecule-1) and VCAM-1 (vascular cell adhesion molecule-1).

Therefore provided herein is a method of modifying a pathogenic angiogenesis in the back of an eye of a patient, the method comprising administering to a patient in need thereof a pathogenic angiogenesis modifying amount of Loteprednol etabonate.

Also provided herein are methods for treating neovascular diseases of the back of the eye comprising administering to a patient in need thereof a therapeutically effective amount of Loteprednol etabonate in a pharmaceutically acceptable vehicle.

### BRIEF DESCRIPTION OF THE DRAWINGS

FIGS. 1A and B are graphical representations of the effect of LE on the expression of VEGF in HREC (human retinal endothelial cells) with (A) or without (B) LPS (lipopolysaccharide, a proinflammatory stimulant that has been shown to induce VEGF or upregulate VEGF induction) activation;

FIGS. 2A and B are graphical representations of the effect of LE on the expression of sVCAM-1 (A) and sICAM-1 (B);

FIG. 3 depicts the physical appearance of 35% LE implants approximately 36 days after being placed in 2% FBS/PBS media;

FIG. 4 depicts the physical appearance of 35% LE implants approximately 36 days after being placed in PBS media;

FIG. 5 depicts the physical appearance of 10% LE implants approximately 36 days after being placed in 2% FBS/PBS media;

FIG. 6 depicts the release rates of the 35% LE implants.

### DETAILED DESCRIPTION

Topical formulations comprising Loteprednol etabonate are commercially available under the trade names ALREX®, LOTEMAX® and ZYLET® from Bausch & Lomb Incorporated and are described in U.S. Pat. Nos. 5,540,930 and 5,747,061 as well as U.S. patent application Ser. No. 10/698,322; the contents of each of which is incorporated by reference herein. Although there is currently no intraocular implants commercially available comprising LE, intraocular implants for the delivery of steroids such as Fluocinolone acetonide are available under the trade name RETISERT® from Bausch & Lomb Incorporated. Another intraocular implant for the delivery of steroids such as LE can be prepared by combining bioerodible PLGA with Loteprednol etabonate and preparing an implant sized and configured to deliver active LE to an intraocular region for an extended period of time.

Neovascular Diseases of the Eye

As noted above, the present invention provides methods for treating neovascular diseases of the back of the eye, including for example, proliferative diabetic retinopathy, retinopathy of prematurity, sickle cell disease, glaucoma associated with angiogenesis and macular degeneration.

Within one aspect of the present invention, methods are provided for treating proliferative diabetic retinopathy, comprising the step of administering to a patient a therapeutically effective amount of a Loteprednol etabonate composition to the eyes, such that the formation of blood vessels is inhibited. Methods of administration can include, for example, use of an intraocular implant containing LE.

In another aspect of the invention, it is contemplated that LE, for example in an implant, would be used the fellow eye when only one eye demonstrates a disease state as prophylaxis for the disease.

Briefly, background diabetic retinopathy is believed to convert to proliferative diabetic retinopathy under the influence of retinal hypoxia. Generally, neovascular tissue sprouts from the optic nerve (usually within 10 mm of the edge), and from the surface of the retina in regions where tissue perfusion is poor. Initially the capillaries grow between the inner modifying membrane of the retina and the posterior surface of the vitreous. Eventually, the vessels grow into the vitreous and through the inner modifying membrane. As the vitreous contracts, traction is applied to the vessels, often resulting in shearing of the vessels and blinding of the vitreous due to hemorrhage. Fibrous traction from scarring in the retina may also produce retinal detachment.

The conventional therapy of choice is panretinal photocoagulation to decrease retinal tissue, and thereby decrease retinal oxygen demands. Although initially effective, there is a high relapse rate with new lesions forming in other parts of the retina. Complications of this therapy include a decrease in peripheral vision of up to 50% of patients, mechanical abrasions of the cornea, laser-induced cataract formation, acute glaucoma, and stimulation of subretinal neovascular growth (which can result in loss of vision). As a result, this procedure is performed only when several risk factors are present, and the risk-benefit ratio is clearly in favor of intervention.

Therefore, within further embodiments of the invention, proliferative diabetic retinopathy may be treated by injection of a Loteprednol etabonate composition and/or intraocular implants comprising LE into the aqueous humor or the vitreous, in order to increase the local concentration of Loteprednol etabonate in the retina. Preferably, this treatment should be initiated prior to the acquisition of severe disease requiring photocoagulation.

Within another aspect of the present invention, methods are provided for treating Retinopathy of prematurity, comprising the step of administering to a patient a therapeutically effective amount of a Loteprednol etabonate composition and/or intraocular implants comprising LE to the eye, such that the formation of blood vessels is inhibited.

Briefly, retinopathy of prematurity is a condition occurring in premature infants who receive oxygen therapy. The peripheral retinal vasculature, particularly on the temporal side, does not become fully formed until the end of fetal life. Excessive oxygen (even levels which would be physiologic at term) and the formation of oxygen free radicals are thought to be important by causing damage to the blood vessels of the immature retina. These vessels constrict, and then become structurally obliterated on exposure to oxygen. As a result, the peripheral retina fails to vascularize and retinal ischemia ensues. In response to the ischemia, neovascularization is induced at the junction of the normal and the ischemic retina.

In 75% of the cases these vessels regress spontaneously. However, in the remaining 25% there is continued capillary growth, contraction of the fibrovascular component, and traction on both the vessels and the retina. This results in vitreous hemorrhage and/or retinal detachment which can lead to blindness. Neovascular angle-closure glaucoma is also a complication of this condition.

As it is often impossible to determine which cases will spontaneously resolve and which will progress in severity, conventional treatment (i.e., surgery) is generally initiated only in patients with established disease and a well developed pathology. This “wait and see” approach precludes early intervention, and allows the progression of disease in the 25% who follow a complicated course. Therefore, within one embodiment of the invention, topical administration of Loteprednol etabonate compositions, as described above may be accomplished in infants who are at high risk for developing this condition in an attempt to cut down on the incidence of progression of retrolental fibroplasia. Within other embodiments, intravitreous injections and/or intraocular implants of a Loteprednol etabonate composition may be utilized. Such methods are particularly preferred in cases of established disease, in order to reduce the need for surgery.

### EXAMPLES

ELISA

HREC were treated with LE (10, 25 and 50 μM) for 3 days. Supernatants were removed and microcentrifuge (Beckman Microfuge R) 1000 RPM for 10 min (4° C. after which they were stored at −70° C. until needed. ELISA kits (R&D systems) for human vascular endothelial growth factor (VEGF), soluble intracellular adhesion molecule (sICAM-1) and soluble vascular cell adhesion molecule (sVCAM-1) were used according to the manufacturer's instructions.

Protein Assay

Protein levels were measured using a Micro BCA protein assay kit (Pierce).

Western Blot Analysis

Cells were treated with LE (0, 10 & 50 μM) for either 3 days or LE (0, 10 & 50 μM) was replenished everyday for 3 days. Cells were lysed and protein levels determined/adjusted (see above). SDS PAGE was performed on cell lysates and then separated proteins were transferred on to a nitrocellulose (NC) paper (Invitrogen). NC paper was blocked with 3% non-fat milk in phosphate buffered saline (PBS) for 1 hour and then probed with specific anti bodies to occludin (Rabbit anti-occludin polyclonal and monoclonal antibodies, Zymed); Goat anti-occludin (Santa Cruz). NC paper was then incubated with secondary horseradish peroxidase conjugated antibody for 1 hour and the bands were visualized using DAB (3,3′-diaminobenzidine) (Vector Laboratories) or cheminluminescence (SuperSignal West Dura, Pierce).

Immunohistochemistry (HREC)

Human retina endothelial cells were seeded onto an 8-well glass chamber and allowed to become 80-90% confluent. Cells were treated with or without LE for 1 hour and then treated with VEGF (20 ng/ml) for 1 hour. HREC were immediately fixed and stained with specific antibodies to occludin and zonula occluden-1 (Zymed). FITC-conjugated secondary antibodies (R&D Systems) were used to visualize tight junction distribution/integrity.

Fabrication of LE/PLGA Sustained Release Implants

10% and 35% Loteprednol etabonate bioerodible implants were hot melt extruded through a lab mixing extruder Dynisco Instruments, Hickory, N.C. using the following formulations:

10% LE Implants

10% LE, Lot # 0208251158B

85% (50:50) PLGA, 0.17 I.V., Lot # D00120

5% (50:50) PLGA, 0.39 I.V., Lot # D01079

The process conditions used were as follows:

Rotor Temp: 72 C

Header Temp: 72 C

Rotor RPM: 40

Line Puller: 70-80 setting

35% LE Implants

35% LE, Lot # 0208251158B

60% (50:50) PLGA, 0.17 I.V., Lot # D00120

5% (50:50) PLGA, 0.39 I.V., Lot # D01079

The process conditions used were as follows:

Rotor Temp: 80 C

Header Temp: 85 C/Second Pass: Header Temp: 83 C

Rotor RPM: 20

Line Puller: 110-130 setting

The extruded LE/PLGA implants were stored in a Petri dish in a nitrogen dry box.

In Vitro Release Study

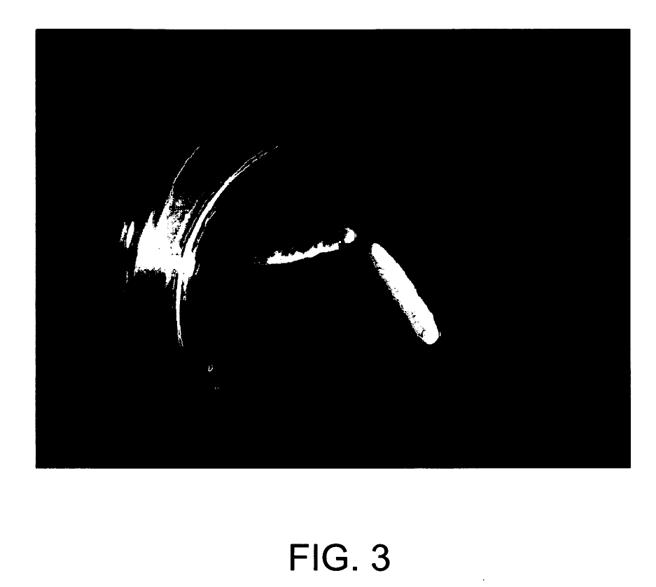

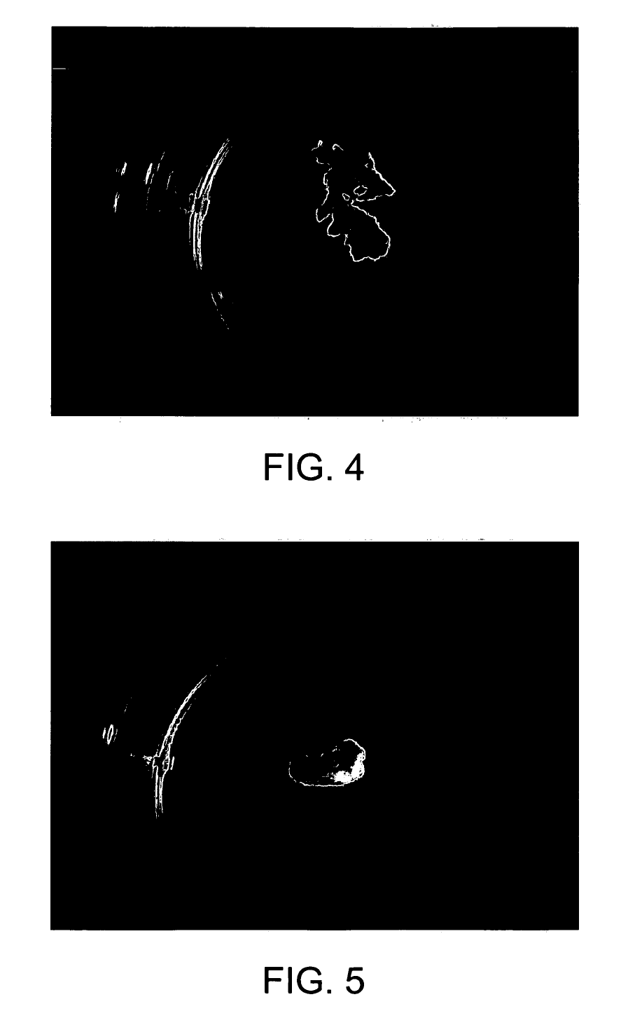

Three implants were placed separately in individual vials containing 3 ml 2% Fetal Bovine Serum (FBS) HyClone, Logan, Utah Phosphate Buffered Saline (PBS) Invitrogen, Carlsbad, Calif. (pH=7.4) and in PBS (pH=7.4) release media. The various media were replaced fully twice a week (every 3 or 4^{th} day). The release of LE from the implants as well as the physical appearance of the devices is shown in the FIGS. 3-6. Implants containing 35% Loteprednol/60% 50:50 PLGA (0.17 IV)/5% 50:50 PLGA (0.39 IV) in PBS and 2% FBS/PBS were initiated. FIG. 3 depicts the 35% LE implants in 2% FBS/PBS implant approximately 36 days after initiation. FIG. 4 depicts the 35% LE implants in PBS media implant approximately 36 days after being placed in PBS media. The implant has severely degraded. The release rate study was abandoned using the PBS model. Implants in PBS and 2% FBS/PBS containing 10% Loteprednol/85% 50:50 PLGA (0.17 IV)/5% 50:50 PLGA (0.30 IV) were initiated. FIG. 5 depicts the 10% LE implant in PBS/2% FBS implant approximately 36 days after initiation. The implant has changed from a rod to a bead shape. The 10% LE implant in PBS completely eroded away during the first week of initiating the implant study. The release rate study for this formulation was abandoned due to the rapid erosion of the implant.

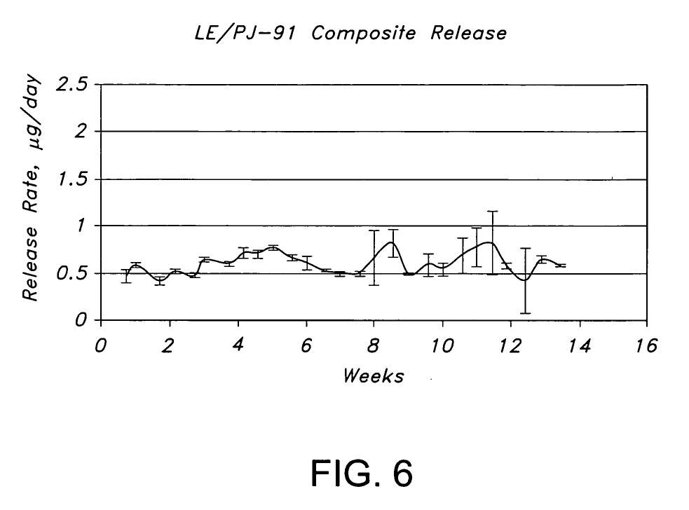

FIG. 6 depicts the release rates of the 35% Loteprednol Etabonate implants. The PLGA bioerodible polymers are known to erode in a bulk erosion fashion. This would imply that as the aqueous media penetrates the polymer the drug present in the matrix would be exposed to this environment. LE, which is known to hydrolyze very rapidly, would be rendered unstable. However, this formulation demonstrated that the drug is released in a sustained manner over a period of at least 13 weeks. This would facilitate the use of LE for various indications such as DME, uveitis, etc. for the posterior segment of the eye using long term sustained release implants. The content uniformity of the 35% LE implants was determined to be: 33.46% (SD:4.9%)

Method and Treatment

A bioerodible device is prepared comprising DL-PLGA copolymers and Loteprednol etabonate. The device is cylindrical in form approximately 0.5 mm diameter by 10 mm in total length. The device is inserted into the vitreous body of the eye in a minimally invasive and sutureless form. The device is designed to provide for the release of active LE to the posterior chamber of the eye for an extended period of time.

Discussion

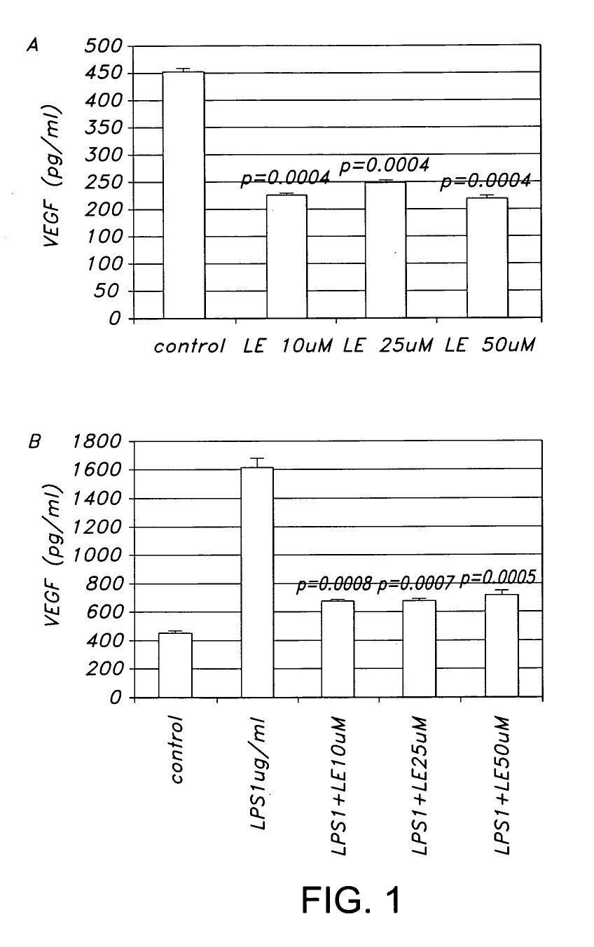

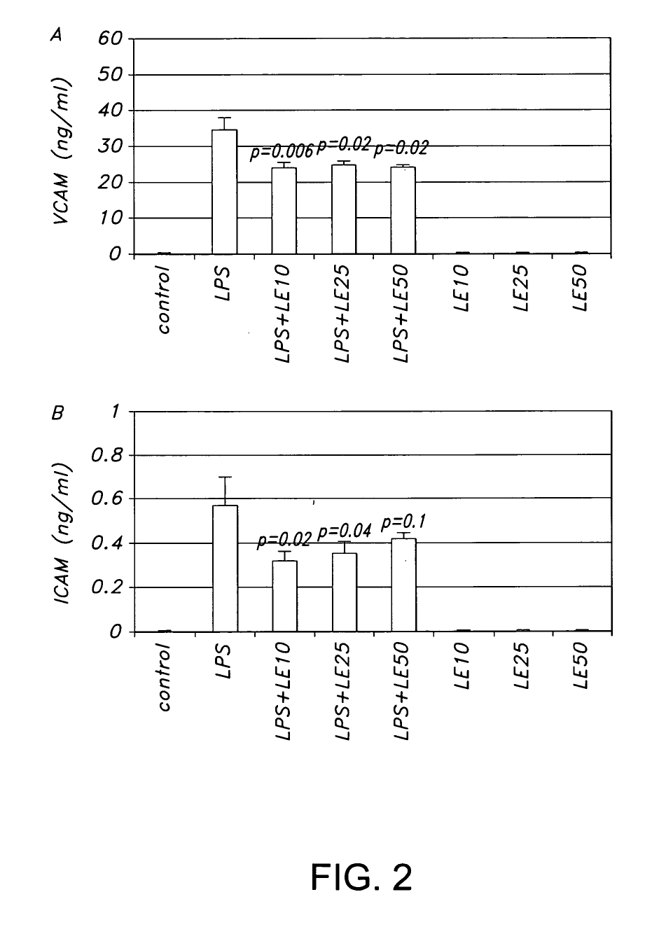

Loteprednol etabonate treatment (0, 10, 25 & 50 μM) of HREC (with or without LPS activation) significantly down regulated the expression of VEGF (p≦0.01) (FIGS. 1A and B), sVCAM-1 (p≦0.05) and sICAM-1 (p≦0.05) (FIGS. 2A and B). There was no apparent cellular toxicity at the highest concentration of LE tested.

Loteprednol etabonate appears to regulate tight junction proteins by inhibiting VEGF-induced redistribution of ZO-1. Photomicrographs were taken to show the effect of LE on ZO-1 in VEGF-treated Human Retinal Endothelial Cells (HREC). HREC stained positively for ZO-1 when no VEGF was present, VEGF-treated cells did not react with ZO-1 and LE at concentration of 10 & 50 μM reversed the effect of VEGF tight junction disruption.

Effect of steroids on vascular leakage and tight junctions has been reported before (Antonetti, D. A., et al.; Journal of Neurochemistry, 2002 80:667-677). In this study LE also inhibited the effect of VEGF-induced disruption of ZO-1 and increased the total content of occludin. However, this modulation of tight junction protein appears to be as a result of LE's steroid effect and not to its metabolites, since daily administration of LE was necessary to maintain its biological effect on occludin.

In conclusion LE is believed to be a good candidate for the treatment of vascular diseases of the eye such as diabetic macular edema and wet AMD.

The claims, as originally presented and as they may be amended, encompass variations, alternatives, modifications, improvements, equivalents, and substantial equivalents of the embodiments and teachings disclosed herein, including those that are presently unforeseen or unappreciated, and that, for example, may arise from applicants/patentees and others.

What is claimed is:

1. A method of modifying a pathogenic angiogenesis in the back of an eye of a patient, the method comprising:
administering to a patient in need thereof a pathogenic angiogenesis modifying amount of Loteprednol etabonate.

2. The method of claim 1 wherein the pathogenic angiogenesis is selected from the group consisting of proliferative diabetic retinopathy, retinopathy of prematurity, sickle cell disease, glaucoma associated with angiogenesis, macular degeneration and combinations thereof.

3. The method of claim 1 wherein the step of administering to a patient in need thereof a pathogenic angiogenesis modifying amount of Loteprednol etabonate is performed by injection of a Loteprednol etabonate composition and/or intraocular implant comprising LE into the aqueous humor or the vitreous of the patient.

4. The method of claim 1 wherein the step of administering to a patient in need thereof a pathogenic angiogenesis modifying amount of Loteprednol etabonate is performed by topically administering a Loteprednol etabonate composition.

5. The method of claim 1 wherein the step of administering to a patient in need thereof a pathogenic angiogenesis modifying amount of Loteprednol etabonate is performed by injection of a Loteprednol etabonate composition and/or intraocular implants comprising LE into the aqueous humor or the vitreous of the patient along with topically administering a Loteprednol etabonate composition.

6. The method of claim 1 wherein the step of administering to a patient in need thereof a pathogenic angiogenesis modifying amount of Loteprednol etabonate is performed to increase the local concentration of Loteprednol etabonate in the retina.

7. The method of claim 1 wherein the step of administering to a patient in need thereof a pathogenic angiogenesis modifying amount of Loteprednol etabonate is performed prior to the acquisition of a disease requiring photocoagulation.

8. A method comprising:
administering to a patient in need thereof a therapeutically effective amount of Loteprednol etabonate in a pharmaceutically acceptable vehicle for treating at least one neovascular disease of the back of the eye.

9. The method of claim 8 wherein the step of administering to a patient in need thereof a therapeutically effective amount of Loteprednol etabonate in a pharmaceutically acceptable vehicle is performed by injection of a Loteprednol etabonate composition and/or intraocular implant comprising LE into the aqueous humor or the vitreous of the patient.

10. The method of claim 8 wherein the step of administering to a patient in need thereof a therapeutically effective amount of Loteprednol etabonate in a pharmaceutically acceptable vehicle is performed by injection of a Loteprednol etabonate composition and/or intraocular implants comprising LE into the aqueous humor or the vitreous of the patient along with topically administering a Loteprednol etabonate composition.

11. The method of claim 8 wherein the neovascular disease of the back of the eye is selected from the group consisting proliferative diabetic retinopathy, retinopathy of prematurity, sickle cell disease, glaucoma associated with angiogenesis and macular degeneration and combinations thereof.

12. The method of claim 8 wherein the neovascular disease of the back of the eye is prophylactically treated by placing an implant in the fellow eye when only one eye demonstrates a neovascular disease state.

13. A method of modifying a pathogenic angiogenesis in the back of an eye of a patient, the method comprising:
administering to a patient in need thereof a pathogenic angiogenesis modifying amount of Loteprednol etabonate.

14. The method of claim 13 wherein the step of administering to a patient in need thereof a pathogenic angiogenesis modifying amount of Loteprednol etabonate is performed by injection of a Loteprednol etabonate composition and/or intraocular implant comprising LE into the aqueous humor or the vitreous of the patient.

15. The method of claim 13 wherein the step of administering to a patient in need thereof a pathogenic angiogenesis modifying amount of Loteprednol etabonate is performed by topically administering a Loteprednol etabonate composition.

16. The method of claim 13 wherein the step of administering to a patient in need thereof a pathogenic angiogenesis modifying amount of Loteprednol etabonate is performed by injection of a Loteprednol etabonate composition and/or intraocular implants comprising LE into the aqueous humor or the vitreous of the patient along with topically administering a Loteprednol etabonate composition.

17. The method of claim 13 wherein the step of administering to a patient in need thereof a pathogenic angiogenesis modifying amount of Loteprednol etabonate is performed to increase the local concentration of Loteprednol etabonate in the retina.

18. The method of claim 13 wherein the step of administering to a patient in need thereof a pathogenic angiogenesis modifying amount of Loteprednol etabonate is performed prior to the acquisition of a disease requiring photocoagulation.

19. A method comprising:
administering to a patient in need thereof a therapeutically effective amount of Loteprednol etabonate in a pharmaceutically acceptable vehicle for treating at least one retinal edema disease of the back of the eye.

20. The method of claim 19 wherein the step of administering to a patient in need thereof a therapeutically effective amount of Loteprednol etabonate in a pharmaceutically acceptable vehicle is performed by injection of a Loteprednol etabonate composition and/or intraocular implant comprising LE into the aqueous humor or the vitreous of the patient.

21. The method of claim 19 wherein the step of administering to a patient in need thereof a therapeutically effective amount of Loteprednol etabonate in a pharmaceutically acceptable vehicle is performed by injection of a Loteprednol etabonate composition and/or intraocular implants comprising LE into the aqueous humor or the vitreous of the patient along with topically administering a Loteprednol etabonate composition.

22. The method of claim 19 wherein the retinal edema disease of the back of the eye is prophylactically treated by placing an implant in the fellow eye when only one eye demonstrates a retinal edema disease state.

23. A method of modifying a pathogenic macular edema in the back of an eye of a patient, the method comprising:
administering to a patient in need thereof a pathogenic macular edema modifying amount of Loteprednol etabonate.

24. The method of claim 23 wherein the step of administering to a patient in need thereof a pathogenic macular edema modifying amount of Loteprednol etabonate is performed by injection of a Loteprednol etabonate composition and/or intraocular implant comprising LE into the aqueous humor or the vitreous of the patient.

25. The method of claim 23 wherein the step of administering to a patient in need thereof a pathogenic macular edema modifying amount of Loteprednol etabonate is performed by topically administering a Loteprednol etabonate composition.

26. The method of claim 23 wherein the step of administering to a patient in need thereof a pathogenic macular edema modifying amount of Loteprednol etabonate is performed by injection of a Loteprednol etabonate composition and/or intraocular implants comprising LE into the aqueous humor or the vitreous of the patient along with topically administering a Loteprednol etabonate composition.

27. The method of claim 23 wherein the step of administering to a patient in need thereof a pathogenic macular edema modifying amount of Loteprednol etabonate is performed to increase the local concentration of Loteprednol etabonate in the retina.

28. The method of claim 23 wherein the step of administering to a patient in need thereof a pathogenic macular edema modifying amount of Loteprednol etabonate is performed prior to the acquisition of a disease requiring photocoagulation.

29. A method comprising:
administering to a patient in need thereof a therapeutically effective amount of Loteprednol etabonate in a pharmaceutically acceptable vehicle for treating at least one macular edema disease of the back of the eye.

30. The method of claim 29 wherein the step of administering to a patient in need thereof a therapeutically effective amount of Loteprednol etabonate in a pharmaceutically acceptable vehicle is performed by injection of a Loteprednol etabonate composition and/or intraocular implant comprising LE into the aqueous humor or the vitreous of the patient.

31. The method of claim 29 wherein the step of administering to a patient in need thereof a therapeutically effective amount of Loteprednol etabonate in a pharmaceutically acceptable vehicle is performed by injection of a Loteprednol etabonate composition and/or intraocular implants comprising LE into the aqueous humor or the vitreous of the patient along with topically administering a Loteprednol etabonate composition.

32. The method of claim 29 wherein the neovascular disease of the back of the eye is prophylactically treated by placing an implant in the fellow eye when only one eye demonstrates a macular edema disease state.

33. A method of modifying a pathogenic macular edema in the back of an eye of a patient, the method comprising:
administering to a patient in need thereof a pathogenic systoid macular edema modifying amount of Loteprednol etabonate.

34. The method of claim 33 wherein the step of administering to a patient in need thereof a pathogenic systoid macular edema modifying amount of Loteprednol etabonate is performed by injection of a Loteprednol etabonate composition and/or intraocular implant comprising LE into the aqueous humor or the vitreous of the patient.

35. The method of claim 33 wherein the step of administering to a patient in need thereof a pathogenic systoid macular edema modifying amount of Loteprednol etabonate is performed by topically administering a Loteprednol etabonate composition.

36. The method of claim 33 wherein the step of administering to a patient in need thereof a pathogenic systoid macular edema modifying amount of Loteprednol etabonate is performed by injection of a Loteprednol etabonate composition and/or intraocular implants comprising LE into the aqueous humor or the vitreous of the patient along with topically administering a Loteprednol etabonate composition.

37. The method of claim 33 wherein the step of administering to a patient in need thereof a pathogenic systoid macular edema modifying amount of Loteprednol etabonate is performed to increase the local concentration of Loteprednol etabonate in the retina.

38. The method of claim 33 wherein the step of administering to a patient in need thereof a pathogenic systoid macular edema modifying amount of Loteprednol etabonate is performed prior to the acquisition of a disease requiring photocoagulation.

39. A method comprising:
administering to a patient in need thereof a therapeutically effective amount of Loteprednol etabonate in a pharmaceutically acceptable vehicle for treating at least one systoid macular edema disease of the back of the eye.

40. The method of claim 39 wherein the step of administering to a patient in need thereof a therapeutically effective amount of Loteprednol etabonate in a pharmaceutically acceptable vehicle is performed by injection of a Loteprednol etabonate composition and/or intraocular implant comprising LE into the aqueous humor or the vitreous of the patient.

41. The method of claim 39 wherein the step of administering to a patient in need thereof a therapeutically effective amount of Loteprednol etabonate in a pharmaceutically acceptable vehicle is performed by injection of a Loteprednol etabonate composition and/or intraocular implants comprising LE into the aqueous humor or the vitreous of the patient along with topically administering a Loteprednol etabonate composition.

42. The method of claim 39 wherein the systoid macular edema disease of the back of the eye is prophylactically treated by placing an implant in the fellow eye when only one eye demonstrates a neovascular disease state.
